(19) United States
(12) Patent Application Publication
(10) Pub. No.: US 2008/0039788 A1
(43) Pub. Date: Feb. 14, 2008

(54) Syringe With Retractable Needle

(76) Inventor: John Riemelmoser, South Australia (AU)

Correspondence Address: KLAUBER & JACKSON, 411 HACKENSACK AVENUE, HACKENSACK, NJ, 07601, US

(21) Appl. No.: 11/596,105

(22) PCT Filed: May 13, 2005
(86) PCT No.: PCT/AU05/00682

§ 371 Date: Nov. 10, 2006

(30) Foreign Application Priority Data
May 14, 2004 (AU) 2004902551

(51) Int. Cl. A61M 5/50

(52) U.S. Cl. 604/110

(57) ABSTRACT

A syringe retraction arrangement including a retaining means (70) mounted on the syringe barrel to retain the syringe within a sleeve assembly (40) on retraction of the hypodermic needle into the sleeve assembly (40). The retaining means (70) includes at least one extension (76, 77), the extension (76, 77) extending outwardly in a radial direction and towards a needle end of the sleeve assembly (40) where he needle (45) resides and wherein the free end of the at least one extension (76, 77) engages an abutment (78, 79) in the sleeve assembly (40) with the abutment (78, 79) acting as a bearing surface for the at least one extension (76, 77) thereby retaining the syringe within the sleeve assembly (40) after retraction.

### FIELD OF THE INVENTION

The present invention relates toga syringe with a needle protection system and in particular to an arrangement that ensures that the needle of the syringe retracts into a container after use to ensure that it is encapsulated thereby preventing a potential needle stick injury. The invention also relates to a single use syringe arrangement.

### BACKGROUND OF THE INVENTION

Needle stick injuries resulting from contact with discarded needles are a well known problem in the medical area. Also the general handling of syringes by medical personnel can expose them to accidental needle stick injuries. In our earlier filed International Patent Application No. PCT/AU03/00346, also entitled “Improved Syringe with Retractable Needle”, whose disclosure is herein incorporated by reference, there is described a syringe retraction arrangement. Referring to FIGS. 1 and 2, this arrangement includes the syringe 2 being of a type having a syringe barrel 3 with a plunger bulb 36 and a plunger arm 21 terminating in a plunger flange 20, the plunger adapted to move in the syringe barrel 3 and a hypodermic needle 45 extending from the syringe barrel 3, the sleeve assembly 40 including a sleeve 41, 42 to receive the syringe 2 with an aperture at one end of the sleeve 41, 42 through which the hypodermic needle 45 extends in use, a pair of ears 55 on the sleeve 41 on the end remote from the aperture, the ears 55 adapted to engage via shoulder region 56 the end 23 of the syringe barrel 3 remote from the hypodermic needle 45 and to release the syringe barrel 3 by disengagement with the end 23 of the barrel when the ears 55 are spread apart and means 37 to move the syringe barrel 3 within the sleeve assembly 40 so as to move the syringe 2 to withdraw the hypodermic needle 45 into the sleeve tube 41, 42 when the end 23 of the syringe barrel 3 is released from the ears 55.

Furthermore, the syringe retraction arrangement includes a guide arrangement 50 mounted onto the syringe barrel 3 which travels in a guide track 48 within the sleeve assembly 40. A detent 60 is included on the guide arrangement 50 which engages with a recess 61 in the sleeve assembly 40 (as best seen in FIG. 2) and thereby retains the guide arrangement 50 and hence the syringe 2 within the sleeve assembly 40 when the syringe needle 45 has been retracted into the sleeve assembly 40 after use.

Although this arrangement is still considered quite satisfactory further developments have been made in order to increase the effectiveness of the retraction mechanism so that the syringe needle is more effectively retained within the sleeve assembly. In some circumstances it has been found that the engagement of detent 60 with recess 61 may be overcome due to flexing of sleeve assembly 40 or alternatively mechanical failure of the detent 60 thereby allowing the retracted syringe 2 to move out of sleeve assembly 40.

It is the object of this invention to provide a syringe retraction arrangement including an improved retaining means capable of preventing the syringe moving outwards after retraction.

### SUMMARY OF THE INVENTION

In a first aspect the present invention accordingly provides a syringe retraction arrangement, said arrangement including:

a syringe, said syringe being of a type having a syringe barrel with a plunger, the plunger comprising a plunger bulb and a plunger arm terminating in a plunger flange, the plunger adapted to move in the syringe barrel and a hypodermic needle extending from the syringe barrel;

a sleeve assembly including a sleeve to receive the syringe with an aperture at one end through which the hypodermic needle extends in use;

a pair of ears located on the sleeve on the end remote from the aperture, said ears adapted to engage the end of the syringe barrel remote from the hypodermic needle and to release the syringe barrel by disengagement with the end of the barrel when the ears are spread apart;

moving means to move the syringe barrel within the sleeve assembly so as to move the syringe to retract the hypodermic needle into the sleeve when the end of the syringe barrel is released from the ears;

retaining means mounted on said syringe barrel to retain the syringe within the sleeve on retraction of said hypodermic needle into said sleeve, wherein said retaining means includes at least one extension, the extension extending outwardly in a radial direction and towards a needle end of said sleeve assembly where the needle resides and wherein the free end of the at least one extension engages an abutment in the sleeve assembly, said abutment acting as a bearing surface for the at least one extension thereby retaining the syringe within the sleeve assembly after retraction.

Preferably, said retaining means includes two opposed extensions and said sleeve assembly includes a corresponding abutment to receive the free ends of each extension.

Preferably, said abutment is located on an inner surface of the sleeve assembly and tapered to cause said extension to further extend outwardly in a radial direction after retraction and on movement of said syringe towards said needle end of said sleeve assembly.

Preferably, said at least one extension is in the form of a resilient prong.

Preferably, said retaining means includes a guide portion, said guide portion adapted to engage with a recess or guide track located on an inner surface of said sleeve assembly thereby forming a guide arrangement for the movement of said syringe barrel in said sleeve assembly.

In a second aspect the present invention accordingly provides a retaining means for retaining a syringe within a sleeve assembly after use of said syringe, said retaining means including:

mounting means to mount said retaining means to said syringe;

at least one extension extending outwardly in a radial direction from said syringe and towards a needle end of said sleeve assembly, wherein in use, said at least one extension engages with said sleeve assembly thereby preventing movement of said syringe towards the needle end of said sleeve assembly.

### BRIEF DESCRIPTION OF THE DRAWINGS

A preferred embodiment of the present invention will be discussed with reference to the accompanying drawings wherein:

FIG. 1 is a perspective view of a prior art syringe retraction arrangement in a partially assembled state;

FIG. 2 is a longitudinal sectional view through A-A of a fully assembled version of the syringe retraction arrangement as illustrated in FIG. 1 after retraction of the syringe;

FIG. 3 is a perspective view of a syringe retraction arrangement according to a preferred embodiment of the present invention;

FIG. 4 is a detailed perspective view of the guide arrangement illustrated in FIG. 3 in a disassembled state;

FIG. 5 is a detailed perspective view of the lower sleeve portion and guide arrangement according to a preferred embodiment of the present invention;

FIG. 6 is a longitudinal sectional view through A-A of a fully assembled version of the syringe retraction arrangement illustrated in FIG. 3 prior to retraction of the syringe;

FIG. 7 is a longitudinal sectional view through B-B of the syringe retraction arrangement illustrated in FIG. 6;

FIG. 8 is a longitudinal sectional view through A-A of awfully assembled version of a syringe retraction arrangement as illustrated in FIG. 3 after retraction of the syringe; and

FIG. 9 is a longitudinal sectional view through B-B of the syringe retraction arrangement illustrated in FIG. 8.

In the following description, like reference characters designate like or corresponding parts throughout the several views of the drawings.

### DESCRIPTION OF PREFERRED EMBODIMENT

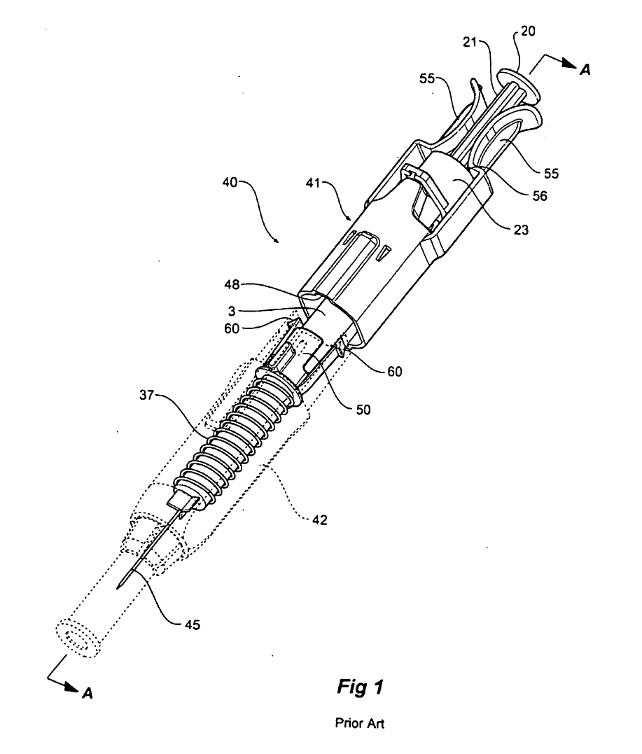

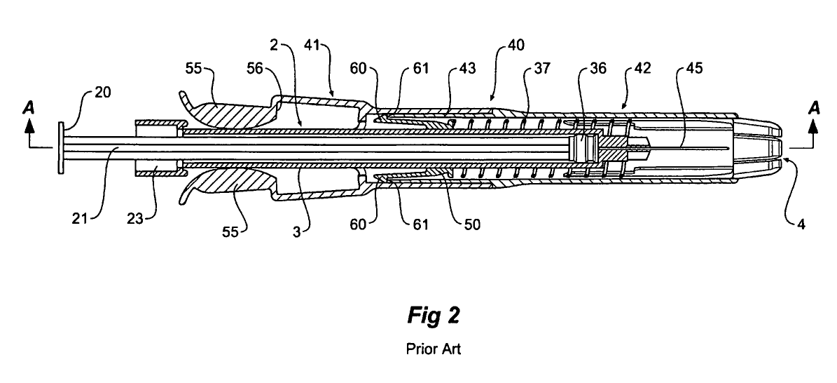

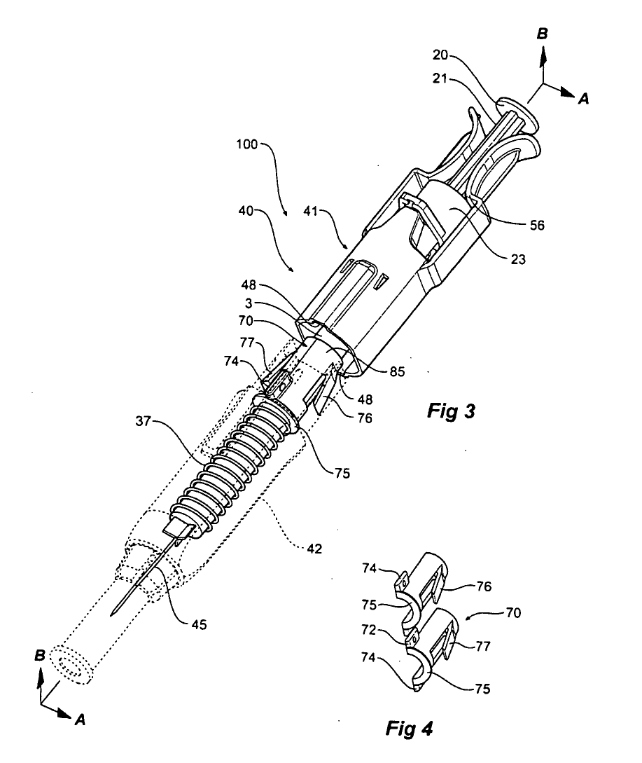

Referring now to FIG. 3 to 5, there is shown a syringe retraction arrangement 100 according to a preferred embodiment of the present invention. Syringe retraction arrangement 100 includes a sleeve assembly 40 including an upper sleeve portion 41 and lower sleeve portion 42 with lower sleeve portion 42 inserted into upper sleeve portion 41 on assembly. With reference to the prior art arrangement depicted in FIGS. 1 and 2, syringe retraction arrangement 100 includes an alternative retaining means which in this preferred embodiment includes guide assembly 70.

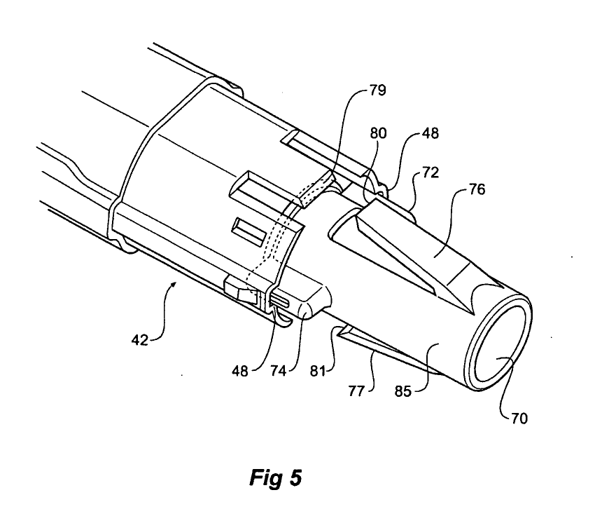

As best seen in FIG. 4, guide assembly 70 has two identical guide portions 72 and 74 which may be clamped around a syringe body 2 and fastened together by ultrasonic welding or other techniques. With reference to FIG. 5, the guide portions 72 and 74 allow the guide assembly 70 to travel along guide tracks 48 located on the inner surface of sleeve assembly 40. The guide assembly 70 includes a pair of opposed extensions in the form of prongs 76, 77 whose tapered ends 80, 81 extend towards the needle end of the syringe 2. Preferably prongs 76 and 77 are manufactured from resilient plastic. Lower sleeve portion 42 includes matching tapered abutments 79, 78 which function to provide a bearing surface against corresponding prong ends 80, 81 should the guide arrangement 70 be moved towards the needle end of sleeve assembly 40 after retraction.

Referring once again to FIG. 3, on assembly of syringe retraction arrangement 100 a spring 37 is placed around the syringe 2 and abuts against surface 75 of the guide assembly 70. Once the prongs 76, 77 are pressed inwardly to contact the main body 85 of the guide assembly 70, the guide assembly 70 and syringe 2 can be mounted into the lower portion 42 of the sleeve assembly 40. The inner surface of the lower portion 42 thus prevents prongs 76, 77 from flexing radially outwardly in this position.

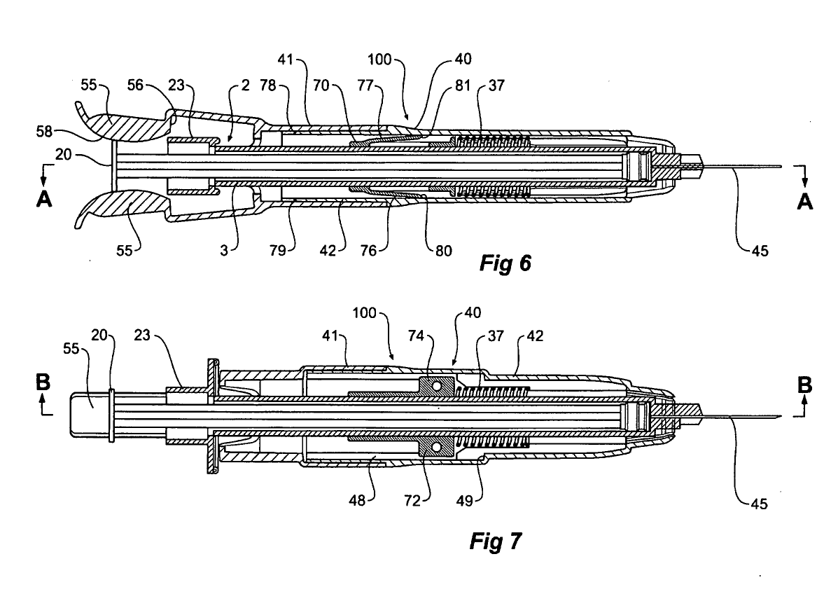

Referring now to FIGS. 6 and 7, there are shown longitudinal sectional views of syringe retraction arrangement 100 as syringe 2 is depressed. In the action of depressing plunger flange 20 into the syringe 2 the plunger flange 20 spreads apart the ears 55 by engaging on inner tapered surfaces 58 which functions to release the upper portion 23 of syringe 2 from the retaining catch of shoulder 56 as the ears 55 are forced apart. Once the upper portion 23 of syringe 2 is relaxed the syringe is forced back due to the resilient action of spring 37 which functions to retract the hypodermic needle 45 into the sleeve assembly 40.

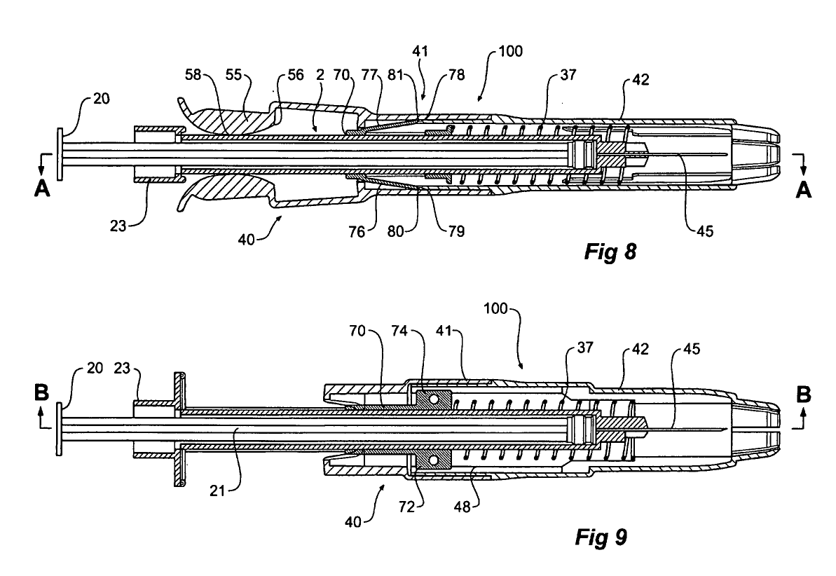

Referring now to FIGS. 8 and 9, there are shown longitudinal sectional views of syringe retraction arrangement 100 after retraction of needle 45. After spring 37 moves guide assembly 70 towards the upper portion 41, prongs 76, 77 through their resilience flex radially outwardly and their tapered ends 80, 81 engage with matching tapered abutments 79, 78 and the inner surface of the upper portion 41. It is sufficient to have a single prong, however a pair of opposed prongs is preferred. In this manner, the prongs 76, 77 prevent the syringe 2 and hence the needle 45 from being re-extended from the sleeve assembly 40 after retraction.

In this configuration any force exerted to move syringe 2 outwards from sleeve assembly 40 thereby re-exposing needle 45 will cause prongs 76, 77 to compress against matching abutments 78, 79. Additionally, prongs 76, 77 will be caused to flex outwardly thereby improving the engagement between the prongs 76, 77 and the corresponding abutments. In the event of a catastrophic failure of prongs 76, 77, the remaining prong sections will engage with the failed prong portions in a wedge configuration thereby preventing further movement of syringe 2 towards the needle end of sleeve assembly 40.

A brief consideration of the above described embodiment will indicate that the syringe retraction arrangement described provides an extremely simple; safety feature for users of syringes and which is effective to improve the retention ability of the arrangement after use of the syringe.

Although a preferred embodiment of the present invention has been described in the foregoing detailed description, it will be understood that the invention is not limited to the embodiment disclosed, but is capable of numerous rearrangements, modifications and substitutions without departing from the scope of the invention as set forth and defined by the following claims.

1. A syringe retraction arrangement, said arrangement including:
a syringe, said syringe being of a type having a syringe barrel with a plunger, the plunger comprising a plunger bulb and a plunger arm terminating in a plunger flange, the plunger adapted to move in the syringe barrel and a hypodermic needle extending from the syringe barrel;
a sleeve assembly including a sleeve to receive the syringe with an aperture at one end through which the hypodermic needle extends in use;
a pair of ears located on the sleeve on the end remote from the aperture, said ears adapted to engage the end of the syringe barrel remote from the hypodermic needle and to release the syringe barrel by disengagement with the end of the barrel when the plunger flange engages with the ears to separate and spread them apart on insertion of the plunger into the syringe barrel;
moving means to move the syringe barrel within the sleeve assembly so as to move the syringe to retract the hypodermic needle into the sleeve when the end of the syringe barrel is released from the ears;
retaining means mounted on said syringe barrel to retain the syringe within the sleeve on retraction of said hypodermic needle into said sleeve, wherein said retaining means includes at least one extension, the extension extending outwardly in a radial direction and towards a needle end of said sleeve assembly where the needle resides and wherein the free end of the at least one extension engages an abutment in the sleeve assembly, said abutment acting as a bearing surface for the at least one extension thereby retaining the syringe within the sleeve assembly after retraction.

2. A syringe retraction arrangement as claimed in claim 1, wherein said retaining means includes two opposed extensions and said sleeve assembly includes a corresponding abutment to receive the free ends of each extension.

3. A syringe retraction arrangement as claimed in claim 1, wherein said abutment is located on an inner surface of the sleeve assembly and tapered to cause said extension to further extend outwardly in a radial direction after retraction and on movement of said syringe towards said needle end of said sleeve assembly.

4. A syringe retraction arrangement as claimed in claim 1, wherein said at least one extension is in the form of a resilient prong.

5. A syringe retraction arrangement as claimed in claim 1, wherein said retaining means includes a guide portion, said guide portion adapted to engage with a recess or guide track located on an inner surface of said sleeve assembly thereby forming a guide arrangement for the movement of said syringe barrel in said sleeve assembly.

6. A syringe retraction arrangement as claimed in claim 1, wherein said moving means is a resilient means.

7. A syringe retraction arrangement as claimed in claim 6, wherein said resilient means includes a spring acting between the sleeve assembly and the syringe barrel.

8. A retaining means as claimed in claim 1, wherein said retaining means is formed as a single plastic moulding and mounting of said retaining means on said syringe barrel includes fastening in places said plastic moulding to said syringe barrel of said syringe by sliding said moulding over said syringe barrel and fastening in place said moulding by an adhesive or an interference fit.

9. A retaining means as claimed in claim 1, wherein said retaining means is formed as a two component piece and mounting of said retaining means on said syringe barrel includes clamping said two component piece around said syringe barrel of said syringe and fastening together said two component piece in the clamped position by ultrasonic welding, or by adhesive or by thermal welding.

10. (canceled)

11. A syringe retraction arrangement as claimed in claim 2, wherein said abutment is located on an inner surface of the sleeve assembly and tapered to cause said extension to further extend outwardly in a radial direction after retraction and on movement of said syringe towards said needle end of said sleeve assembly.

12. A syringe retraction arrangement as claimed in claim 11, wherein said at least one extension is in the form of a resilient prong.

13. A syringe retraction arrangement as claimed in claim 12, wherein said retaining means includes a guide portion, said guide portion adapted to engage with a recess or guide track located on an inner surface of said sleeve assembly thereby forming a guide arrangement for the movement of said syringe barrel in said sleeve assembly.

14. A syringe retraction arrangement as claimed in claim 13, wherein said moving means is a resilient means.

15. A syringe retraction arrangement as claimed in claim 14, wherein said resilient means includes a spring acting between the sleeve assembly and the syringe barrel.

16. A retaining means as claimed in claim 13, wherein said retaining means is formed as a single plastic moulding and mounting of said retaining means on said syringe barrel includes fastening in places said plastic moulding to said syringe barrel of said syringe by sliding said moulding over said syringe barrel and fastening in place said moulding by an adhesive or an interference fit.

17. A retaining means as claimed in claim 13, wherein said retaining means is formed as a two component piece and mounting of said retaining means on said syringe barrel includes clamping said two component piece around said syringe barrel of said syringe and fastening together said two component piece in the clamped position by ultrasonic welding, or by adhesive or by thermal welding.

18. A syringe retraction arrangement as claimed in claim 3, wherein said abutment is located on an inner surface of the sleeve assembly and tapered to cause said extension to further extend outwardly in a radial direction after retraction and on movement of said syringe towards said needle end of said sleeve assembly.

19. A syringe retraction arrangement as claimed in claim 18, wherein said at least one extension is in the form of a resilient prong.

20. A syringe retraction arrangement as claimed in claim 19, wherein said retaining means includes a guide portion, said guide portion adapted to engage with a recess or guide track located on an inner surface of said sleeve assembly thereby forming a guide arrangement for the movement of said syringe barrel in said sleeve assembly.
